(19) United States
(12) Patent Application Publication
(10) Pub. No.: US 2012/0209363 A1
(43) Pub. Date: Aug. 16, 2012

(54) HOT AND COLD THERAPY DEVICE

(75) Inventors: Riley J. Williams, III, New York, NY (US); Roy Swan, JR., New York, NY (US)

(73) Assignee: R2T2 SOLUTIONS LLC, Sag Harbor, NY (US)

(21) Appl. No.: 13/247,113

(22) Filed: Sep. 28, 2011

Related U.S. Application Data
(63) Continuation-in-part of application No. 13/025,077, filed on Feb. 10, 2011.

(51) Int. Cl. A61F 7/00

(52) U.S. Cl. 607/114

(57) ABSTRACT

A thermal therapy device including a hollow roller having a non-uniform cross-section along its length and a thermally conductive external surface. Two end caps, one on each end of the hollow roller, which, in combination with the thermally conductive external surface of the hollow roller, collectively define a thermal-medium-receiving reservoir and a central axis about which the hollow roller may rotate. A pair of pins, one on each of the end caps, are longitudinally aligned along the central axis.

### CROSS REFERENCE TO RELATED APPLICATION

This application is a continuation-in-part of co-pending U.S. patent application Ser. No. 13/025,077 filed on Feb. 10, 2011, the entirety of which is incorporated herein by reference.

### BACKGROUND

1. Field

This disclosure relates generally to devices for treating a desired region of a patient's body, and, more particularly, to a device for providing thermal therapy to an affected area on a patient's body.

2. Background

Common injuries, such as (but not limited to) joint sprains, muscle strains, shin splints, and bursitis of the knee, elbow, shoulder, leg, hip, trunk or ankle, often propagate an inflammatory response. Treatment of such injuries and other painful, inflammatory disorders of the muscles, ligaments, tendons, bones and joints may include the application of localized heat and/or cold temperature to the afflicted area of the patient's body. For instance, cryotherapy is an effective modality for the reduction of pain caused by inflammation. Cold material may be placed against the affected area of a patient's body, thereby absorbing heat from the body through thermal conduction. This may trigger a variety of beneficial physiological effects. For example, the application of cold temperature to the afflicted area may produce a numbing effect, reducing the pain experienced by the injured patient. It may also trigger a constriction, which, by restricting the blood flow to the affected area, may reduce swelling. Other beneficial effects may be produced, such as a reduction in local metabolic rate, or relief from uncontrolled muscle spasms. In addition, cold therapy is a known anti-inflammatory modality that is commonly applied to reduce the pain, swelling and discomfort associated with local joint or soft-tissue inflammation.

Similarly, massage is well known for its soothing effects on injured or sensitive areas of the body. The beneficial effects of a massage may be combined with cryotherapy and/or heat therapy to provide superior treatment relative to either cryotherapy or massage alone.

### BRIEF SUMMARY

In one aspect of this disclosure, a thermal therapy device is disclosed. The thermal therapy device has a support frame including two lateral supports and two spaced apart support arms. At least a first of the support arms has, on a distal end thereof, a pair of prongs, each having a length, the pair of prongs being spaced apart so as to define an opening therebetween and dimensioned to removably receive a pin inserted along the prong-length direction with the spacing being at least a pin-width wide. The thermal therapy device further includes a hollow roller having a non-uniform cross-section along its length and a thermally conductive external surface, two end caps, one on each end of the hollow roller, which, in combination with the thermally conductive external surface of the hollow roller, collectively define a thermal-medium-receiving reservoir and a central axis about which the hollow roller may rotate, a first pin coupled to one of the end caps and a second pin, coaxially aligned with the first pin, coupled to the other end cap. At least one of the first pin or the second pin is configured to be removably received within the opening between the prongs along the prong-length direction such that:

i) the axis and prong length direction collectively define a plane when the first pin is within the opening between the prongs and the second pin is within an opening of a second of the support arms, ii) when the first and second pins are within their respective openings, a thermal medium is within the reservoir, and the external surface is supported on a therapeutic target at a point on the plane, the hollow roller can rotate freely within the support frame when the support frame is moved perpendicular to the plane, and iii) when the support frame is moved, along the plane, away from the first pin, the first pin will change from a position within the opening to a position beyond the distal end of the prongs.

In another aspect of this disclosure, an alternative thermal therapy device is disclosed. The thermal therapy device includes a hollow roller having a non-uniform cross-section along its length and a thermally conductive external surface, two end caps, one on each end of the hollow roller, which, in combination with the thermally conductive external surface of the hollow roller, collectively define a thermal-medium-receiving reservoir and a central axis about which the hollow roller may rotate. In addition, one of the two end caps has a first configuration in which, a thermal medium, comprising one of a refrigerant gel, sodium acetate, or a mixture of water and ammonium nitrate, can be transferred to or from the reservoir, and, a second configuration, in which a connection with the hollow roller is formed through which the thermal medium cannot pass. The thermal therapy device also includes a pair of pins, one on each of the end caps, when the at least one end cap is in the second configuration, the pins are longitudinally aligned along the central axis.

The foregoing has outlined rather generally the features and technical advantages of one or more embodiments of this disclosure in order that the following detailed description may be better understood. Additional features and advantages of this disclosure will be described hereinafter, which may form the subject of the claims of this application.

### BRIEF DESCRIPTION OF THE DRAWINGS

This disclosure is further described in the detailed description that follows, with reference to the drawings, in which:

FIG. 1 is a front plan view of an example hot and cold therapy device;

FIG. 2 is a side elevation view of the hot and cold therapy device of FIG. 1;

FIG. 3 is front top plan view of an illustrative flexible belt for use with the hot and cold therapy device of FIG. 1;

FIG. 4 is a front plan view of the hot and cold therapy device roller of FIG. 1 assembled to the flexible belt of FIG. 3;

FIG. 5 is a side elevation view of the assembled hot and cold therapy device roller and flexible belt of FIG. 4;

FIG. 6 is a front cross-sectional view of the hot and cold therapy device taken across line A-A of FIG. 2;

FIG. 7 is a side elevation view of another embodiment of the hot and cold therapy device;

FIG. 8 is a front elevation view of another embodiment of the hot and cold therapy device;

FIG. 9 is a side elevation view of the hot and cold therapy device of FIG. 8;

FIGS. 10A-10C illustrate some representative, non-limiting, example alternative roller shapes;

FIGS. 11A-11C are, respectively, a front, bottom and side view of an alternative configuration frame support and roller;

FIGS. 12A-12D are, respectively, a front, top, side and perspective view of a frame support similar to the frame support of FIGS. 11A-11C;

FIG. 13 is an exploded view of one example implementation roller assembly that can be used with one or more of the examples described herein;

FIG. 14 shows alternative enlarged areas of a cross sectional view of one example therapy device as described herein;

FIGS. 15A and 15B are, respectively, a side and front view of another example end cap 1308 as described above;

FIG. 16 is an example cross section, taken along line Y-Y of FIG. 15B, of another example end cap;

FIG. 17 is yet another example cross section, taken along line Y-Y of FIG. 15B, of another example end cap;

FIG. 18 is an end view of a representative example end cap-receiving end of a roller body;

FIG. 19 is an example cross section of a portion of one example roller taken at line Z-Z of FIG. 18;

FIG. 20 is an alternative example cross section of a portion of one example roller taken at line Z-Z of FIG. 18;

FIG. 21 is another alternative example cross section of a portion of one example roller taken at line Z-Z of FIG. 18; and

FIG. 22 illustrates, in simplified form, another example implementation of a therapy device such as described herein having an aspect ratio of about 3:1 or more and an alternative example handle.

### DETAILED DESCRIPTION

This application discloses a hot and cold therapy device 100 that may be used to apply a hot or cold massage therapy to an area of the body of a patient. The hot and cold therapy device 100 includes a roller that may be filled with a heated or chilled thermal medium (e.g., liquid) to enable application of a hot or cold message therapy.

Additionally, the hot and cold therapy device 100 may be disassembled, which may be advantageous for a number of reasons. First, it allows a user to heat or chill the thermal medium within the roller without heating or chilling the entire device. Second, it may allow a user to utilize a number of different rollers having various shapes, textures and other characteristics beneficial to a particular application, such as conforming to the shape of the portion of the body to be treated. Switching between rollers may be performed quickly and easily. Third, it allows the roller to be releasably connected to a flexible strap, so that the roller may be held in place against the desired portion of the body. This may allow hands free application of continuous, static hot or cold pressure to that area of the body.

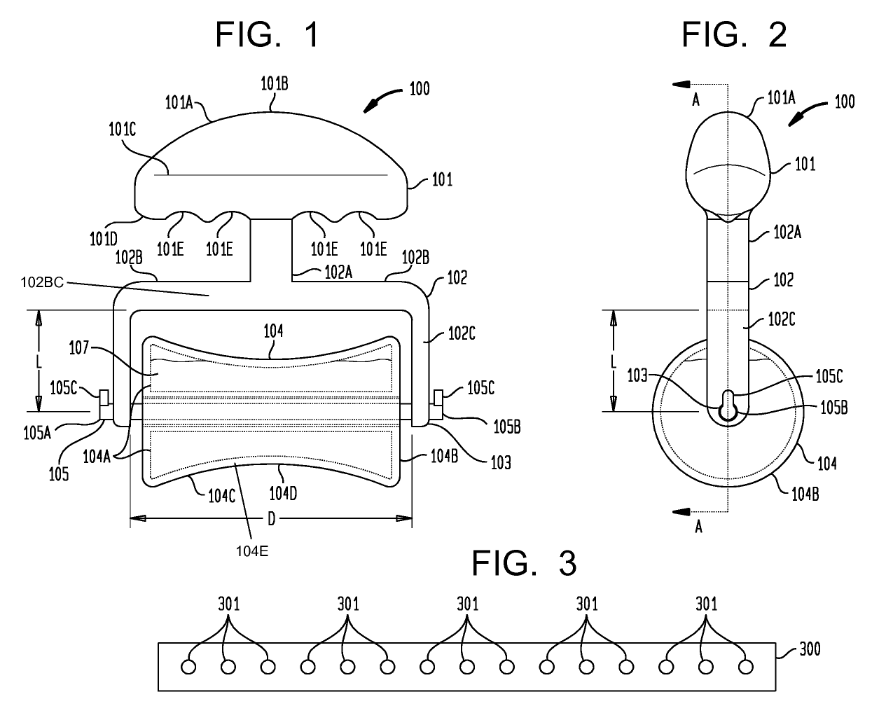

FIGS. 1, 2 and 6 illustrate an example, fully assembled hot and cold therapy device 100. Implementations of the hot and cold therapy device 100 include a handle 101, a frame 102 and a roller 104. In some example implementations, the handle 101 is an ergonomically shaped handle designed for fitting comfortably in a user's hand and is preferably formed of a durable, lightweight, but comfortable to hold material, such as (but not limited to) plastic, rubber, metal, wood, or plastic coated with a rubber-like material. In this embodiment, the hot and cold therapy device 100 is held in the user's palm facing down against the affected area of the patient's body. This handle 101 is ergonomically shaped to comfortably support this kind of “palm down” grip by the user. Implementations of the handle 101 may, for example, have a semi-circular rounded top curvature 101A. The top curvature 101A starts from a midpoint 101B and slopes gently down to a peripheral edge 101C. A bottom segment 101D of handle 101 may include a plurality of indentations 101E that conform to the user's fingers when the user grips the hot and cold therapy device 100 with a palm down grip. Alternatively, the ergonomic handle 101 may be sufficiently tall and/or wide so that the user's hand and fingers do not contact the bottom surface 101D, in which case, the bottom surface 101D may be flat.

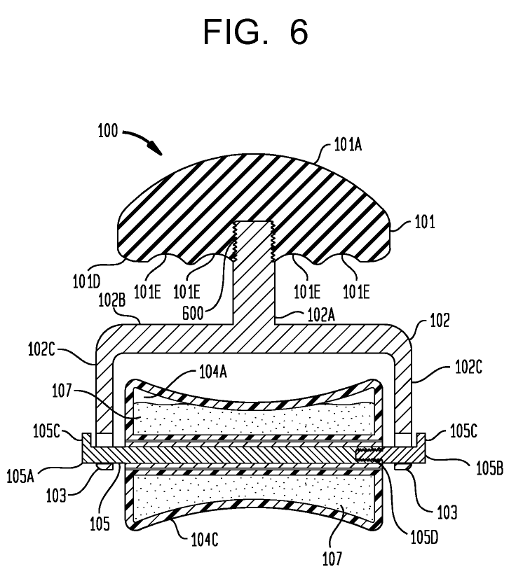

The ergonomic handle 101 and frame 102 may be connected or otherwise assembled together in any conventional manner. The handle 101 and frame 102 may be manufactured as a single component using, for example, conventional injection molding techniques. Alternatively, as best illustrated in FIG. 6, the handle 101 and frame 102 may be separate components in which, for example, frame 102 may abut or extend into the bottom surface 101D of the body of the ergonomic handle 101. Mating helical threads 600 may be provided on both the frame 102 and handle 101 to releasably connect both components to one another via rotation of one component relative to the other. Alternatively, glue, epoxy, soldering, welding, or one or more mechanical fasteners may be utilized to releasably or permanently connect the handle 101 to the frame 102.

Frame 102 defines the main body of the therapy device 100. Frame 102 may be made from any suitable material that is strong enough to withstand the forces typically exerted on the therapy device 100 without suffering structural failure. For example, the frame 102 may be made from plastic, metal or wood. The frame 102 may have any desired cross-section or desired combination of cross sections. As depicted in the drawings, the frame 102 has a compact, predominantly square or rectangular cross section. The edges are preferably rounded or smoothed to eliminate sharp edges that may injure a user or patient and, possibly, to present an aesthetically pleasing finish for the user. Alternatively, circular, ovular, L-shaped, C-shaped or even irregular cross-sections may be utilized as desired.

With some implementations, the frame 102 includes a neck 102A, at least one lateral member 102B and at least one supporting arm 102C projecting or otherwise extending from the terminal end of the lateral member 102B. In the example embodiment of FIG. 1, the frame 102 includes a frame support 102BC made up of a pair of lateral members 102B extending laterally from the neck 102A, and a pair of spaced apart supporting arms 102C projecting or otherwise extending from the terminal end of the lateral members 102B. The neck 102A is either releasably or permanently connected to the either or both of the frame support 102BC and/or a handle, such as the ergonomic handle 101 of FIG. 1. Depending upon the specific implementation, the handle 101 and frame 102 may be integrally formed as a single unit or as separate components joined in any conventional manner using, for example, mating male and female helical threads 600, glue, epoxy or some other adhesive, soldering, welding, or one or more other types mechanical fasteners or fastening approaches.

As shown in FIG. 1, the neck 102A projects substantially perpendicularly from the lower surface 101D of ergonomic handle 101 and branches laterally into at least one lateral member 102B. In one example embodiment, a pair of lateral members 102B extend in opposing directions from the neck 102A in a generally perpendicular direction to the neck, thereby forming a single horizontal structure that follows a generally parallel path relative to the plane of the lower handle surface 101D.

Each lateral member 102B transitions into a support arm 102C, which is the portion of frame 102 or frame support 102BC releasably connected to the roller 104. Each support arm 102C extends generally parallel to each other, generally perpendicular to the lateral member 102B and includes an opening 103 for releasably and rotatably connecting the roller 104 to the frame support 102BC (described further below). The support arms 102C should be spaced apart by a distance D that is sufficient to accommodate the width of the roller 104 and permit the roller to rotate freely relative to the frame 102 without interference from lateral members 102B. Similarly, the distance L between the underside of the lateral members 102B and the central axis of the opening 103 in the support arms 102C should be sufficient to accommodate the maximum radius of the roller 104 and permit the roller to rotate freely when mounted in the frame 102. It is understood that the angle between the support arms 102C and the lateral members 102B may be any desired angle provided the angles and distances D and L are sufficient to allow the roller 104 to rotate freely when mounted within the frame 102.

The roller 104 is used to apply hot or cold massage therapy to the desired area of the patient's body. The roller 104 is made from a thermally conductive material (at least in the portion 104E of the body 104C which will apply the heat or cold therapy), such as (but not limited to) metal, an appropriate thermally conductive plastic, rubber or other sufficiently firm thermally conductive material(s). As best illustrated in FIGS. 1 and 6, the roller 104 includes a hollow chamber or reservoir 104A configured for receiving and storing a heatable, chillable and/or freezable heat transferable medium 107. For use, the heat transferable medium 107 may be a non-toxic liquid, fluid, gel, gas, refrigerant or other substance that can be heated and/or cooled to a desired temperature and maintained within the roller 104.

The heat transferable medium 107 may be inserted into, and then sealed, within the hollow chamber 104A during manufacture of the roller 104 or shortly prior to use. Alternatively, one or more sealable openings (for example, as described herein) may be provided to allow for transfer of the heat transferable medium 107 into or out of the roller 104 preferably via a surface or location that does not contact the patient during use. Of course, alternatively, the roller 104 can be configured to allow for transfer of the heat transferable medium 107 to or from the reservoir 104A at a location that would normally contact the patient during use provided the location can be closed in such a manner that the heat transferable medium 107 will not undesirably leak out and contact the patient in a manner that could cause injury or significant discomfort.

As noted above, any suitable heat transferable material or substance may be utilized as the heat transferable medium 107. For example, hollow chamber 104A may be filled with water. The water may then be refrigerated or frozen, thereby providing a reservoir containing a low temperature heat transferable material within roller 104 for a cold therapy massage. Similarly, an oil or other viscous fluid can be used as the heat transferable medium 107, particularly an oil that can retain heat or remain cool relative to its surroundings for an extended period of time. Alternatively, more exotic substances may be used. For example, a refrigerant gel may be utilized in place of water as the heat transferable medium 107 within the reservoir 104A of roller 104. Refrigerant gels are common materials typically utilized in ice packs. Refrigerant gels may include water and sodium carboxymethyl cellulose, which is a nontoxic, noncarcinogenic mixture that provides temperatures colder than those attainable by water ice, allowing it to remain cold or frozen longer in room temperature environments.

Other heat transferable substances may be utilized as heat transferable medium 107 to produce beneficial effects. For example, a mixture of water and ammonium nitrate may be utilized as the heat transferable medium 107. Such mixtures are commonly utilized in “cold packs,” and may be utilized here as well. When water and ammonium nitrate are combined, the resulting mixture generates an endothermic reaction, greatly lowering the temperature of the solution (and thus, the roller 104), which may be beneficial for applying a cold massage therapy with the roller 104.

Alternatively, a solution of sodium acetate (dissolved into its water of crystallization) may be utilized as heat transferable medium 107. Such solutions are commonly utilized in “heating pads,” “hand warmers” and other portable heat emitting devices. A mechanism can be provided within the roller 104 to allow a nucleation center to be formed within the solution, causing the solution to begin crystallizing. The crystallization process is exothermic, and thus emits heat, which may be beneficial for applying a heated massage therapy with the roller 104. The sodium acetate solution may thereafter be reused by boiling the solution within the roller 104 to dissolve the sodium acetate trihydrate crystals.

The roller 104 may generally have a cylindrical (but not necessarily uniform) form, with circular end sections 104B and a cylindrical body 104C. The circular sections 104B may each present a generally flat surface. For example, as shown, the cylindrical body 104C includes a concave slope that begins at or near a circumferential edge of a section 104B, which may then gradually decrease down toward a midpoint 104D (or another location) of roller 104. Therefore, in this example, cylindrical body 104C has the smallest cross-sectional diameter relative to the cross-sectional diameters along its entire length (up to a maximum diameter possessed by circular sections 104B) at or near midpoint 104D. This approach may be used to present a more ergonomic shape to the roller 104, thereby enabling superior application of pressure and heat or cold therapy to the affected region of the patient's body.

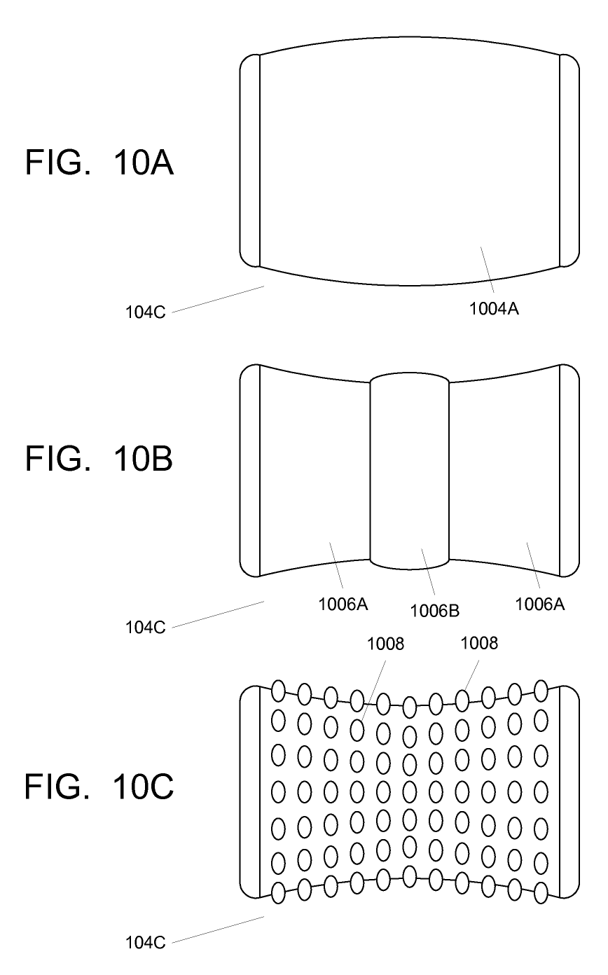

Other roller shapes may be utilized as desired to conform to the particular area of the patient's body that is being treated. FIGS. 10A-10C illustrate some representative, non-limiting, example alternative roller shapes. For example, a roller 104 with a less concave (or even flat) cylindrical body 104C may be utilized for massaging flatter portions of the body, such as the patient's abdominal or back bones or muscles. Alternatively, as shown in FIG. 10A, a roller 104 may be formed with a raised or convex cylindrical body 1004A, cresting at or near its midpoint (or another location) between the two circular sections 104B. Such a roller 104 may be used, for example, to target a very specific region of the patient's body. Any advantageous shape may be utilized for roller 104 as desired, including, for example as shown in FIG. 10B, a combination of concave 1006A and convex 1006B configurations for the cylindrical body 104C. Additionally, small shapes, textures or other surface modifications 1008, for example as shown in FIG. 10C, may be added to the cylindrical body 104C to affect a different feeling during the massage or improve the hot or cold therapy.

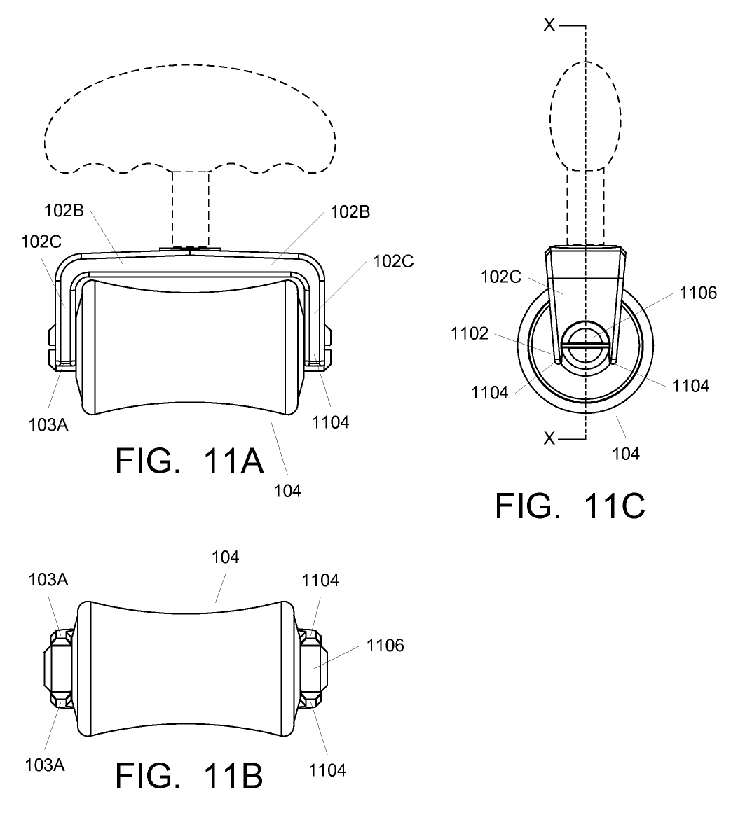

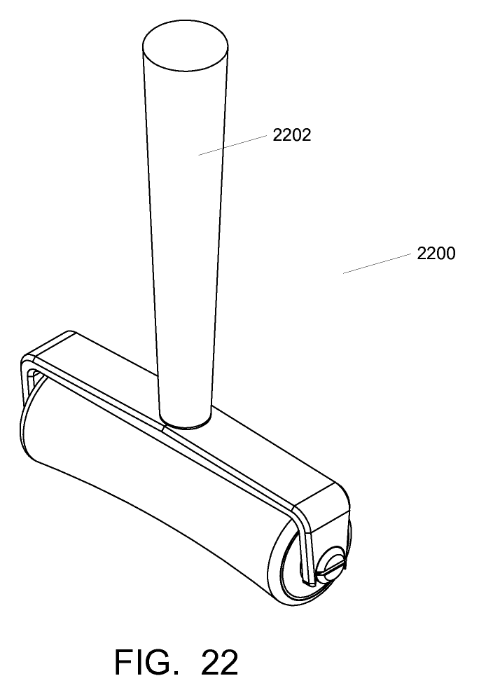

Similarly, the aspect ratio of the roller 104 can be modified. For example, as shown in FIG. 22, the aspect ratio of length to largest diameter is about 3:1 or more, whereas the aspect ratio of the roller in FIG. 11 is about 1.5:1 or more and the aspect ratio of the roller of FIG. 1 is somewhere between the two.

Referring to FIGS. 1 and 6, an axle or shaft 105 may be utilized to provide an axis for the rotation of the roller 104 relative to the frame 102. The roller 104 may include a longitudinally extending opening or bore and, optionally, a longitudinally extending sleeve or journal 106 may be press fit within the longitudinally extending opening of the roller. The roller 104 can be configured so that an axle 105 may be inserted into the longitudinally extending opening (or into the sleeve or journal 106 if provided) and extend longitudinally through the roller 104 into respective longitudinally aligned openings 103 formed in the support arms 102C of frame 102. Each end of the axle 105 typically extends beyond the longitudinally aligned openings 103 of the frame 102 and generally includes one or more protrusions 105C, which serve both as a stop to prevent the axle 105 from sliding out of the frame 102 and, optionally, as a means for releasably connecting the roller 104 to a flexible belt or band 300 if the roller 104 is to be held statically in place against the intended portion of the body (as will be described further below with respect to FIGS. 3-5). The roller 104 may also be manufactured such that there is no clearly defined central axis or, alternatively, an axle 105 is unnecessary because the material used to make the roller is formed in such a manner that the side connectors (105A and B, C) project directly and contiguously from the sides of the roller, leaving a greater volume within the roller 104 than would otherwise be available with a configuration that uses an axle 105.

To facilitate convenient and easy attachment/detachment of the roller 104 to the frame 102, at least one end of the axle 105 may include a threaded opening 105D for releasably engaging to mating threads on a threaded fastener or coupling 105B. While the fastener 105B is illustrated in FIG. 6 as having male threads and the axle 500 as having a corresponding female threaded opening 105D, it is understood that the axle could alternatively include the male threads and the fastener could include a corresponding female threaded opening. The threaded fastener 105B has a body portion with a diameter that is approximately the same size as the diameter of the axle 105 so that the threaded fastener may be inserted through an opening 103 in the support arm 102C of frame 102. The threaded fastener 105B can include a protrusion 104F at its terminal end in the same manner as described above.

Therefore, during assembly of the hot and cold therapy device 100, the axle 105 can be inserted through an opening 103 within one of the support arms 102C and then through the longitudinally extending opening (or sleeve or journal 106 if provided) of roller 104 positioned between the support arms 102C of frame 102. The threaded fastener 105B may then be inserted through the co-aligned opening 103 of the opposing support arm 102C and threaded into the threaded opening 105D at the end of the axle 105, thereby releasably and rotatably connecting roller 104 to frame 102. The roller 104 may be removed from the frame 102 in a similar, but opposite manner, by unscrewing the threaded fastener 105B from the end of the axle 105, after which the axle may be removed from the longitudinally extending opening (or sleeve or journal 106 if provided) of the roller 104 and the opening 103 within the support arm 102C through which it passes.

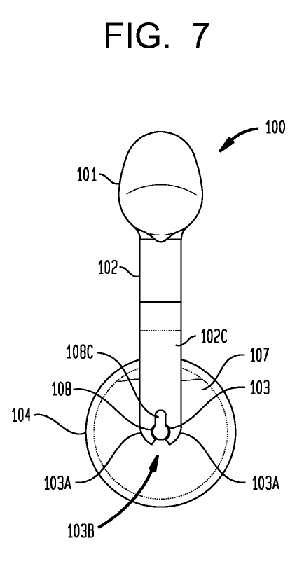

In an alternative embodiment illustrated in FIG. 7, the roller 104 may include a pin or cylindrical member 108 extending longitudinally along a central axis from each end of the roller 104 in lieu of the axle 105. Alternatively, the cylindrical member 108 can simply be a part of the axle 105 near its end. With the embodiment of FIG. 7, a pair of pliable jaws 103A located in each supporting arm 102C of frame 102 is provided that partially encloses and defines the opening 103 in the supporting arms 102C. The pliable jaws 103A are separated by a small gap 103B through which the pin or cylindrical member 108 extending from the roller 104 may be forcibly introduced, so that the pair of jaws 103A flex a sufficient amount to allow the pin or cylindrical member 108 to pass through the gap 103B into the opening 103 and then be partially surrounded by the jaws 103A. Once the pin or cylindrical member 108 is inserted within the opening 103 surrounded by the jaws 103A, the jaws snap back or elastically return to their original configuration to retain the pin or cylindrical member 108 within the opening 103 partially surrounded by the jaws to thereby releasably and rotatably connect the roller 104 to the support arms 102C. The roller 104 may similarly be removed from the frame 102 by applying a force on the pin or cylindrical member 108 sufficient to cause the pair of pliable jaws 103A to flex away from one another and permit the pin or cylindrical member 108 to pass through the gap 103B between the jaws. The pliable jaws 103A may be formed in the support arms 102C in a conventional manner, such as, for example, during a molding process for manufacturing frame 102. It is understood that other mechanisms for releasably and rotatably connecting the roller 104 to the frame 102 may be utilized as well. The pin or cylindrical members 108 extending longitudinally from the roller 104 may also include one or more protrusions 108C (similar to protrusions 105C described above) to releasably connect the roller 104 to a flexible strap 300 when the roller 104 is to be held statically in place against the intended portion of the body (as described with respect to FIGS. 3-5).

FIG. 3 illustrates the flexible strap or tape 300 that may be utilized in conjunction with the roller 104 to apply static hot or cold pressure against an area of the patient's body. Flexible strap or tape 300 preferably includes a plurality of openings 301 centrally aligned along the strap's longitudinal axis. The flexible strap or tape 300 may be a flexible strap, belt, band, bandage or tape made of any suitable material, such as (but not limited to) cloth athletic tape, a woven elastic bandage or fabric, or the like.

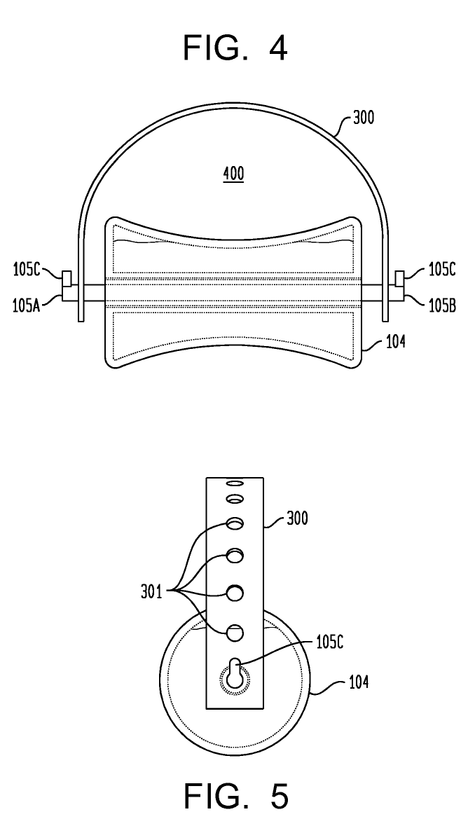

FIGS. 4 and 5 illustrate an example in which the flexible strap 300 is releasably connected to the roller 104, in lieu of the frame support 102BC, in order to maintain the roller in a static position against an area of a patient's body to be treated for some period of time. The flexible strap 300 may be releasably connected to the roller 104 by inserting the end 105A of the axle 105 and the threaded fastener 105B into respective openings 301 within the strap 300. The protrusions 105C extending from the ends 105A of the axle 105 and the threaded fastener 105B extend beyond the circumference of openings 301 to thereby prevent the strap 300 from unintentionally disconnecting from the roller 104. When assembled in this manner, the flexible strap 300 and roller 104 define an open region 400. The roller and strap assembly may therefore be wrapped around a body part, such as an arm or leg, in which the body part is positioned within the loop 400. The roller and strap assembly may then be tensioned against the body part by selecting the appropriate opening 301 such that sufficient tension exists to statically maintain the roller 400 in place against the body part to be treated. In this manner, pressure and hot or cold therapy may be statically applied to the designated area.

Prior to use, the heat transferable medium 107 may be heated or cooled to about the desired temperature by, for example, pre-heating or cooling the heat transferable medium 107 prior to introducing it into the reservoir or, if already in the reservoir, by placing the thermally conductive roller in a suitable heating or cooling device, such as (but not limited to) an oven, microwave, refrigerator or freezer. Once the roller 400 (and the heat transferable medium 107) have reached the desired temperature, the roller may either be releasably and rotatably connected to the frame 102 so that a user may apply the hot or cold therapy to an area of the patient's body by massaging the area with the thermally conductive roller. Alternatively, the thermally conductive roller 400 may be used without the frame support 102BC and positioned against the area of the patient's body to be treated and the flexible strap 300 may be wrapped about the intended area and releasably attached to the roller to apply static hot or cold therapy to that area.

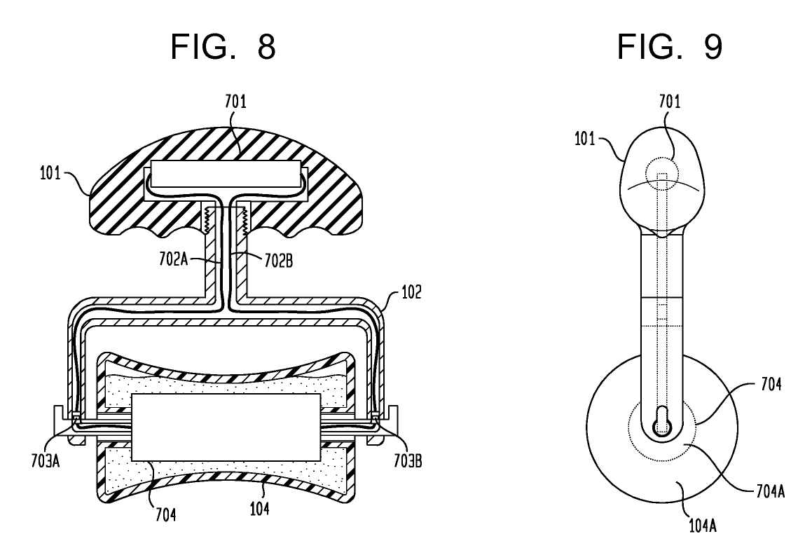

FIG. 8 illustrates another example embodiment of the hot and cold therapy device 700, with FIG. 9 showing a side elevation view of the hot and cold therapy device of FIG. 8. As shown, the therapy device 700 is optionally equipped with an electrically powered mechanism 704 for providing additional effects as part of the massage therapy. For example, the electrically powered mechanism 704 may include a vibratory motor that vibrates within the roller 104, enabling the addition of vibration to a massage therapy. Alternatively or additionally, the electrically powered mechanism 704 may optionally include an electrically operated heating element for heating the heat transferable medium 107 within the roller 104 and/or the roller itself, allowing one to potentially alter or control the temperature of a cold massage, or to apply (or transition to) a hot massage.

The handle 101 preferably houses the power source 701 supplying power to the electrically powered mechanism 704. Standard battery solutions may be utilized as the power source 701. For example, the power source 701 may be removable in the form of one or more standard replaceable batteries or rechargeable batteries. Alternatively, the power source 701 may be permanently installed in the handle 101. Where the power source is rechargeable (e.g., rechargeable battery(ies)), recharging of the power source 701 may be achieved via an externally attachable charging cable. In yet another embodiment, a motion-charged battery may be used, allowing motion from an applied massage to at least partially recharge the battery.

In one example implementation, such as shown, the power source 701 is electrically connected to the electrical mechanism 704 via an electrical circuit 702a and 702b housed within the frame 102. Electrical contacts or connectors 703a and 703b complete the electrical circuit to the electrically powered mechanism 704 housed within the body of roller 104. The electrical contacts or connectors 703a and 703b are designed so as to provide continuous continuity even as the roller 104 rotates relative to frame 102 using any of multiple conventional known approaches.

Alternatively, the power source can be configured to supply power to the electrical mechanism 704 in a non-contact manner using, for example, capacitive or inductive coupling.

In this manner, the roller 104 may be removed from the frame 102. Optionally, a separate chamber may be included in the body of the roller 104 to house the electrically powered mechanism 704, thereby ensuring proper function of the mechanism 704 by segregating the heat transferable medium 107 from the mechanism 704 and, where applicable, the electrical contacts 703a and 703b.

It should be understood and appreciated that any electrical mechanism 704 described herein can optionally be incorporated into any configuration of rollers 104 described herein.

FIGS. 11A, 11B and 11C are, respectively, a front, bottom and side view of an alternative configuration frame support 102BC and roller 104. As shown, this configuration is similar to the configuration of FIG. 7. However, this configuration differs from FIG. 7 in two respects. First, with this configuration, the frame support 102BC is separate and discrete from the neck 102A. Second, unlike FIG. 7, with this configuration, the distal end 1102 of at least one of the support arms 102C is configured with a pair of prongs 1104 which are spaced apart from each other at spacing sufficient to removably receive an end section of an axle 105 or a pin 1106 but in a way that does not hold the axle 105 or pin 1106 within the opening if the point where the lateral support 102B transitions to the support arm 102C of the frame support 102BC is moved away from the axle 105 or pin 1006 along the plane defined by the section line X-X of FIG. 10C. In other words, the spacing between the prongs along their length is always, in the area that can receive the relevant part of axle 105 or pin 1006, wider than that relevant part. As a result, with this configuration, the axle 105 or pin 1006 will generally only be constrained within the support arm 102C when a part of the external surface of the roller 104 (to the distal side of the prongs 1104) is supported on a surface and, in combination, the frame support 102BC and roller 104 are moved in a direction other than along the plane as described immediately above.

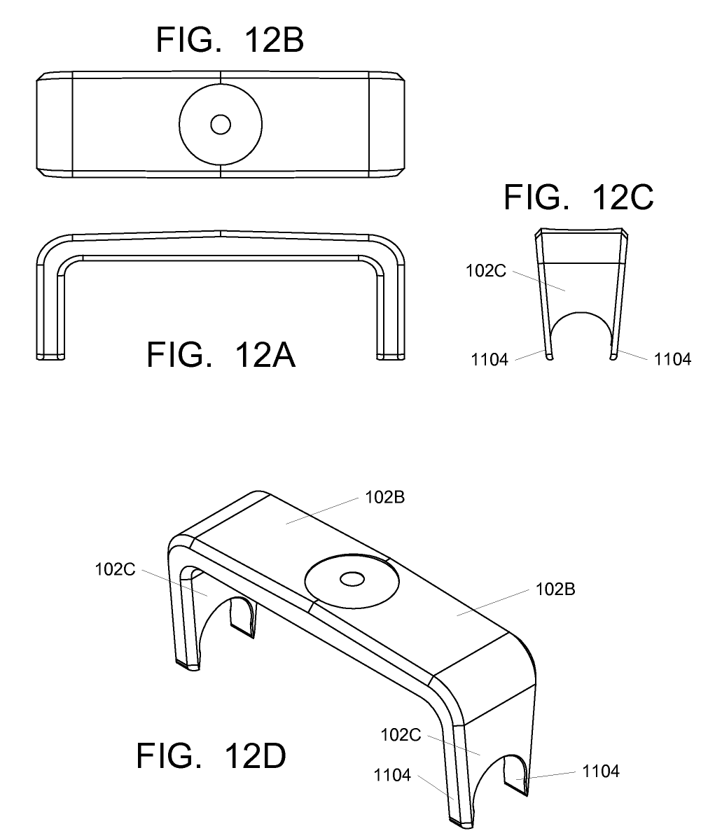

FIGS. 12A-12D are, respectively, a front, top, side and perspective view of a frame support 102BC similar to that of FIGS. 11A-11C. In FIGS. 12C-12D, the prongs 1004 and an example of their axle or pin-receiving spacing can be more clearly seen. As can also be seen, the frame support 102BC of FIGS. 12A-12D differs from that of FIGS. 11A-11C in that, with FIGS. 11A-11C, one of the support arms 102C includes a pair of jaws 103A, and the other support arm 102C includes the prongs 1004 as described immediately above, whereas the frame support 102BC of FIGS. 12A-12D are configured with prongs 1004 on the distal end of each of the support arms 102C.

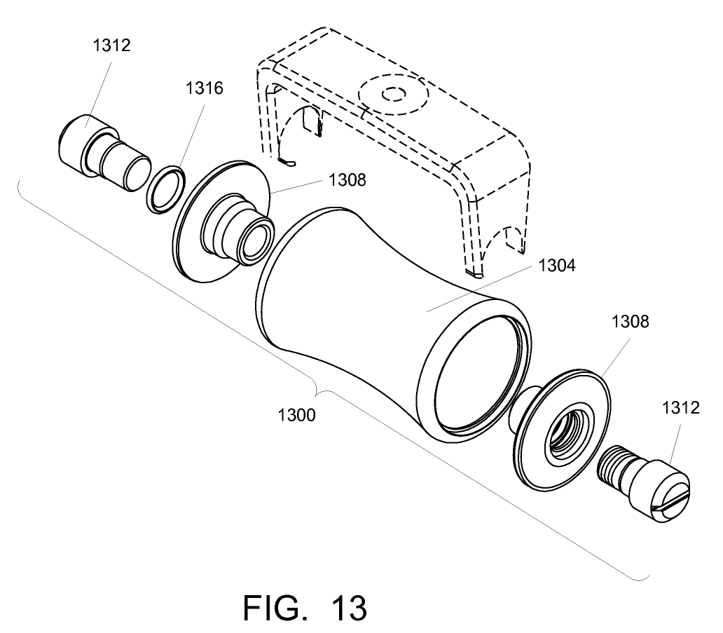

FIG. 13 is an exploded view of one example implementation roller assembly 1300 that can, but need not, be used as a roller 104 in one or more of the examples described herein. The assembly 1300 is made up of a thermally conductive hollow cylindrical roller body 1304 (similar to the roller body 104C), a pair of end caps 1308 (one on each end of the roller body 1304), and a pair of pins or cylindrical members 1312. As will be described in greater detail below, there are different possible configurations for one or both end caps 1308 and their respective pins 1312. As a result, the assembly 1300 may also include one or more sealing mechanisms 1316, for example, washers, gaskets or O-ring seals. When assembled, the roller body 1304 and end caps 1308 collectively define the reservoir 104A for holding the heat transferable medium 107.

The roller assembly 1300 is specifically configured to allow for the heat transferable medium 107 to be inserted into or removed from the reservoir 104A at some point after manufacture. Specifically, depending upon the particular implementation embodiment, the roller assembly 1300 is configured, in different example implementations, such that one or both of the end caps 1308 and/or one or both pins or cylindrical members 1312 can be removed to provide access to the reservoir 104A. In this manner, different types of heat transferable medium 107 can be used with the same device. By way of illustrative, non-limiting example, as shown in FIG. 13, the end caps 1308 are configured to not be removable after manufacture, but the pin 1312 on the left works in conjunction with an O-ring seal 1316 such that the two act as a removable pressure-fit stopper to allow for the heat transferable medium 107 to be inserted into or removed from the reservoir 104A.

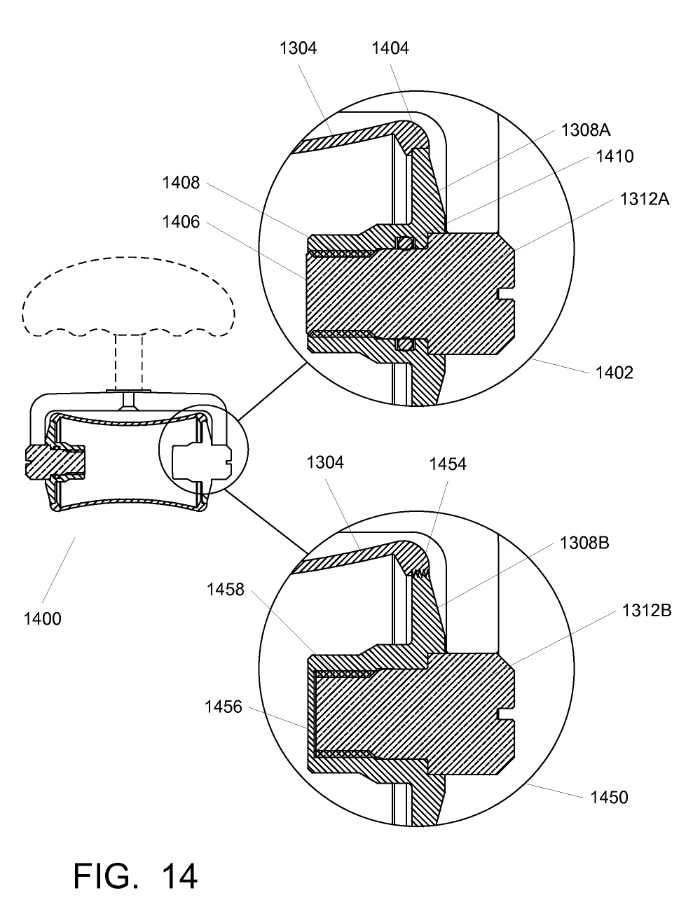

Other representative example alternative configurations for one or more of the pins or cylindrical members 1312 can be seen in FIG. 14 in the alternative enlarged areas 1402, 1450 of the cross sectional view of one example therapy device 1400 as described herein.

With the example of the first enlarged area 1402, one end cap 1308A is configured to be essentially the same size as the periphery of the end of the roller body 1304 so that the end cap forms a junction 1404 that will not allow the heat transferable medium 107 to pass through. Depending upon the particular implementation example, this junction can be permanent connection, for example, formed by gluing, welding or other appropriate form of permanent bonding or joining, or it can merely be a pressure-fit type connection that allows for the end cap to be removed and/or replaced through suitable application of force. As shown, the junction 1404 is a permanent connection.

In order to get the heat transferable medium 107 into or out of the reservoir with this example configuration, the end cap 1308A contains a passageway 1406 that goes all the way through the end cap 1308A.

Thus, with this configuration, the pin or cylindrical member 1312A is fitted with screw threads 1408 that allow for the pin or cylindrical member 1312A to be securely, but releasably, attached to the end cap 1308A via the threads. In addition, to ensure that the heat transferable medium 107 cannot leak out during use, the end cap 1308A also includes an integral sealing mechanism 1410, for example in this case, a gasket or O-ring.

With the example of the second enlarged area 1450, one end cap 1308B is configured with threats that mate with complementary threads on the surface of an end of the roller body 1304 so as to form a releasable screw-type junction 1454 between the two that is sufficiently tight to ensure that the heat transferable medium 107 cannot leak out during use. Since the end cap is removable, the end 1456 of the end cap 1308B in this configuration is solid (i.e., there is no opening through this end cap 1308B). The pin or cylindrical member 1312B of this configuration is, dependent upon the particular implementation, designed to be held in place through any suitable approach 1458, including, for example, screw threads, a deformable member, or a locking mechanism, to name a few.

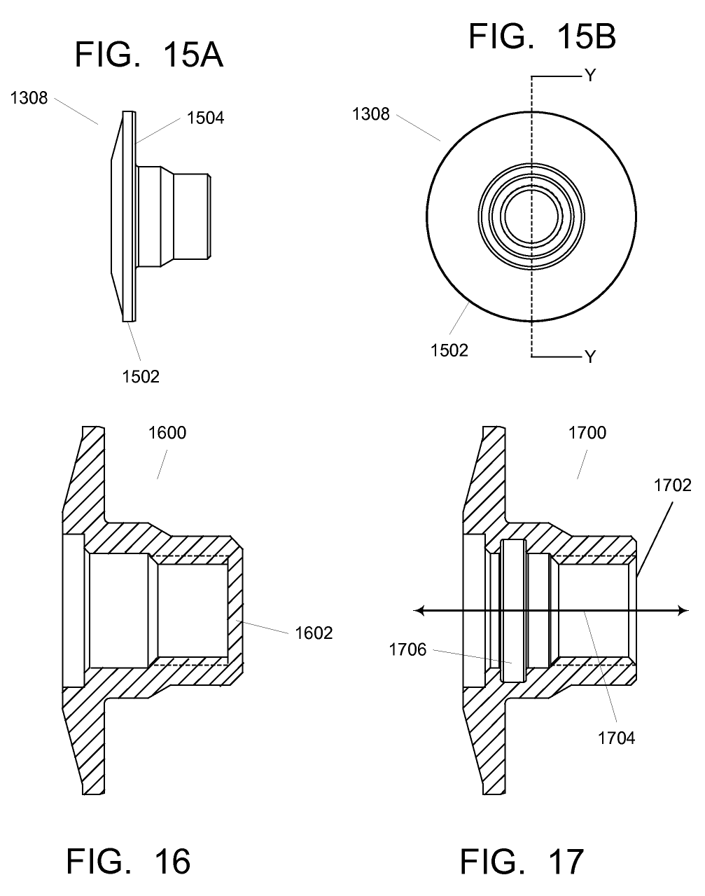

FIGS. 15A and 15B are, respectively, a side and front view of another example end cap 1308 as described above. Note that, with this example, the periphery 1502 of this end cap 1308 includes a locking element 1504, for example a slight indent or raised ridge, to facilitate forming of a locking connection with a complementary mechanism on the periphery of the end of a roller body 104C.

FIG. 16 is an example cross section, taken along line Y-Y of FIG. 15B, of another example end cap 1600. Note that this end cap is similar to the end cap 1308B of FIG. 14 in that is configured such that it includes a solid end 1602.

FIG. 17 is yet another example cross section, taken along line Y-Y of FIG. 15B, of another example end cap 1700. As shown in FIG. 17, this end cap is similar to the end cap 1308A of FIG. 14 in that it does not have a solid end 1702 but, rather, is designed with a through-passage 1704 that can be used to pass the heat transferable medium 107. In addition, this end cap 1700 includes an area 1706 to accommodate a sealing or locking mechanism that can releasably constrain a pin or cylindrical member. Note that such a configuration could also include screw threads if desired.

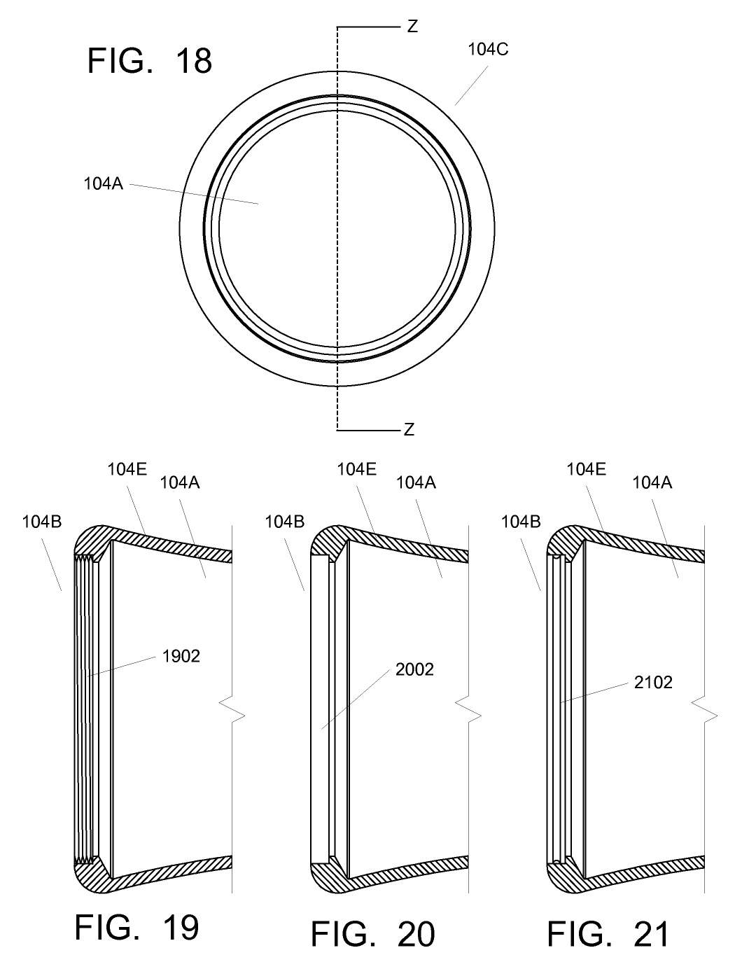

FIG. 18 is an end view of a representative example end cap-receiving end 104B of a roller body 104C.

FIG. 19 is an example cross section of a portion of one example roller taken at line Z-Z of FIG. 18. With this example roller, the end 104B includes threads 1902 designed to allow a complementary threaded end cap (not shown) to be removably attached thereto.

FIG. 20 is an alternative example cross section of a portion of one example roller taken at line Z-Z of FIG. 18. With this example roller, the end 104B is smooth 2002 and designed to closely correspond to a smooth end cap so that the end cap can be permanently attached or form a pressure-fit removable attachment of sufficient closeness to prevent leakage during use as described above.

FIG. 21 is another alternative example cross section of a portion of one example roller taken at line Z-Z of FIG. 18. With this example roller, the end 104B includes one or more a circumferential raised or recessed feature(s) 2102, in this case a raised semicircular bump) designed to mate with a complementary feature of an end cap (not shown) to form a locking mechanism that allows them to be removably connected to each other in a secure and leak-resistant manner.

FIG. 22 illustrates, in simplified form, another example implementation of a therapy device 2200 such as described herein having an aspect ratio of length to largest diameter of about 3:1 or more and an elongated handle 2202 (which can be separate, integral, or formed as merely an elongated neck) instead of the ergonomic handle of, for example, FIG. 1. As shown, the elongated handle 2202 is tapered, but could interchangeably be straight or incorporate/include material(s) or one or more features to make it more comfortable or ergonomic, and it could be solid or hollow, in whole or part, to affect the weight or cost of manufacture, the specific construction of the handle being unimportant to understanding the device or how it is used.

Having described and illustrated the principles of this application by reference to one or more preferred embodiments, it should be apparent that the preferred embodiment(s) may be modified in arrangement and detail without departing from the principles disclosed herein and that it is intended that the application be construed as including all such modifications and variations insofar as they come within the spirit and scope of the subject matter disclosed.

What is claimed is:

1. A thermal therapy device comprising:
a support frame including
two lateral supports, and
two spaced apart support arms,
at least a first of the support arms having on a distal end thereof a pair of prongs, each having a length, the pair of prongs being spaced apart so as to define an opening therebetween and dimensioned to removably receive a pin inserted along a prong-length direction with the spacing being at least a pin-width wide;
a hollow roller having a non-uniform cross-section along its length and a thermally conductive external surface;
two end caps, one on each end of the hollow roller, which, in combination with the thermally conductive external surface of the hollow roller, collectively define a thermal-medium-receiving reservoir and a central axis about which the hollow roller may rotate;
a first pin coupled to one of the end caps and a second pin, coaxially aligned with the first pin, coupled to the other end cap, at least one of the first pin or the second pin being configured to be removably received within the opening between the prongs along the prong-length direction such that
i) the axis and prong length direction collectively define a plane when the first pin is within the opening between the prongs and the second pin is within an opening of a second of the support arms,
ii) when the first and second pins are within their respective openings, a thermal medium is within the reservoir, and the external surface is supported on a therapeutic target at a point on the plane, the hollow roller can rotate freely within the support frame when the support frame is moved perpendicular to the plane, and
iii) when the support frame is moved, along the plane, away from the first pin, the first pin will change from a position within the opening to a position beyond the distal end of the prongs.

2. The thermal therapy device of claim 1, wherein the non-uniform cross-section is generally concave along its length.

3. The thermal therapy device of claim 1, wherein a thermal medium is within the reservoir and wherein the thermal medium comprises:
a refrigerant gel, a mixture of water and ammonium nitrate, or sodium acetate.

4. The thermal therapy device of claim 1, further comprising an electrical mechanism within the hollow roller.

5. The thermal therapy device of claim 4, wherein the electrical mechanism includes at least one of: a heating element or a vibrating element.

6. The thermal therapy device of claim 4, wherein the prongs are rigid such that, when the support frame is moved, along the plane, away from the first pin, the first pin will change from the position within the opening to the position beyond the distal end of the prongs without requiring flexure of the prongs.

7. The thermal therapy device of claim 1, wherein at least one of the end caps is removable.

8. The thermal therapy device of claim 7, wherein both of the end caps are removable.

9. The thermal therapy device of claim 7, wherein an outer periphery of the at least one removable end cap is dimensioned relative to a peripheral end portion of the hollow roller such that, when the at least one removable end cap is positioned on the hollow roller, a juncture is formed which will not allow a thermal medium in the reservoir to pass from the reservoir through the juncture.

10. The thermal therapy device of claim 7, wherein the at least one end cap is affix-able to the peripheral end via a compression connection.

11. The thermal therapy device of claim 7, wherein the at least one end cap is affix-able to the peripheral end via a threaded connection.

12. The thermal therapy device of claim 1, further comprising a handle coupled to the support frame.

13. The thermal therapy device of claim 1, wherein the hollow roller has an aspect ratio of length to height of at least about 1.5 to 1.

14. A thermal therapy device comprising:
a hollow roller having a non-uniform cross-section along its length and a thermally conductive external surface;
two end caps, one on each end of the hollow roller, which, in combination with the thermally conductive external surface of the hollow roller, collectively define a thermal-medium-receiving reservoir and a central axis about which the hollow roller may rotate,
wherein one of the two end caps has a first configuration in which, a thermal medium, comprising one of a refrigerant gel, sodium acetate, or a mixture of water and ammonium nitrate, can be transferred to or from the reservoir, and, a second configuration, in which a connection with the hollow roller is formed through which the thermal medium cannot pass; and
a pair of pins, one on each of the end caps, when the at least one end cap is in the second configuration, the pins are longitudinally aligned along the central axis.

15. The thermal therapy device of claim 14, further comprising a frame support comprising a pair of lateral supports and two support arms extending therefrom, the two support arms each having pin-receiving openings on a distal end thereof.

16. The thermal therapy device of claim 15, wherein the pins are rotatably located within the pin-receiving openings.

17. The thermal therapy device of claim 14, further comprising a flexible strap having a first hole therein coupled to one of the pins and a second hole coupled to the other of the pins.

18. The thermal therapy device of claim 14, wherein, in the second configuration, the at least one end cap is coupled to the hollow roller by a screw connection.

19. The thermal therapy device of claim 14, wherein, in the second configuration, the at least one end cap is coupled to the hollow roller by a pressure-fit connection.

20. The thermal therapy device of claim 14, wherein at least one of the pins is connectable to its respective end cap by one of:
a screw thread, an O-ring seal or a pressure-fit connection.
